(19) United States
(12) Patent Application Publication
(10) Pub. No.: US 2010/0145235 A1
(43) Pub. Date: Jun. 10, 2010

(54) Device for fixing at least one fibrous strip to a body part, and method for the production of said strip

(76) Inventors: Dirk David Goldbeck, München (DE); Tobias Happel, Berlin (DE); Benjamin L'Hénoret, Toulouse (FR); Helmut Nowsch, Regensburg (DE); Daniel Sommer, Ulm (DE)

Correspondence Address: King & Spalding LLP, 401 Congress Avenue, Suite 3200, Austin, TX, 78701, US

(21) Appl. No.: 12/513,036

(22) PCT Filed: Oct. 24, 2007
(86) PCT No.: PCT/EP2007/061439

§ 371 Date: Feb. 16, 2010

(30) Foreign Application Priority Data
Nov. 2, 2006 (DE) 102006051742.3
Jun. 15, 2007 (DE) 102007027722.0

(51) Int. Cl. A61B 5/103

(52) U.S. Cl. 600/595

(57) ABSTRACT

A device (V) for fixing at least one fibrous strip (FB) to a body part (K), wherein the fibrous strip comprises at least one fibre (F) embodied as a fibre-optic bending sensor, has at least one fixing unit (BE) which respectively has (a) a fixing part (BM) for applying part of the fibrous strip to the body part, and (b) at least one fixing loop (BS) for guiding the fibrous strip along an axis of a three-dimensional space. The fixing loop can be fixed to the body part.

Device for fixing at least one fibrous strip to a body part, as well as a method for the production of a device for fixing two fibrous strips in a parallel arrangement to a body part

The invention relates to a device for fixing at least one fibrous strip to a body part, as well as to a method for production of a device for fixing two fibrous strips in a parallel arrangement to a body part.

The practice of using fiber optic bending sensors for measuring bending angles, for example by bending a finger joint, is known, see [1]. In such cases the fiber optic bending sensor has one or more sensitive zones which are handled such that, as a result of the bending of the fiber optic bending sensor, a light attenuation varies as a function of a bending angle.

This technology has also long been used for measuring flexions and torsions of parts of the body, such as the flexion of a finger joint, in humans and animals for example.

The problem arising in such cases is to provide a device for fixing at least one fiber optic bending sensor in such a way that said sensor delivers reliable measurement results despite a change in a body surface to be measured.

This object is achieved by the features of the independent claims. Developments are to be taken from the dependent claims.

The invention relates to a device for fixing at least one fibrous strip to a body part, with the fibrous strip comprising at least one fiber which is embodied as a fiber optic bending sensor, with the device including at least one fixing unit and the respective fixing unit (a) comprising a fixing means for applying a part of the fibrous strip to the body part and (b) at least one fixing loop for guiding the fibrous strip along an axis of a three-dimensional space, with the fixing loop able to be fixed to the body part.

This device allows reliable measurement results since the fiber on a moving body part can both follow the movements of the body part and can also not slip as a result of the movements such that a measurement of the movements is falsified by the slipping. The fixing means in particular ensures that the fiber assumes the same position again after the body part has moved back into its original position.

If the at least one fixing loop and/or the fixing means of the at least one fixing unit are attached to an expandable fixing support, especially a sticking plaster material, with the extensible fixing support able to be fixed to the body part by an adhesive, the device can be fixed to the body part in a simple manner and removed from the body part again, providing a precise fixing with a simple correction facility. This development also has the advantage of the extensible fixing material also being able to follow changes in the length of the body part, through bending or stretching of the back for example, without the device being displaced so that the measurement results become unreliable.

In a development the device comprises a single fixing unit with a single fixing means and two or more fixing loops, the body part is a back, the fixing means is arranged above the fixing loops, especially in the upper area of the back, and the fixing loops are arranged below one another on the back.

The development represents a cost-effective implementation of the device.

In an alternate development the device comprises two or more fixing units, with each of the fixing units comprising a single fixing means, at least one fixing loop and a fibrous strip with at least one fiber, and with the fixing units being arranged below one another on the back.

This means that the device is embodied so as to be well able to follow large changes of the back, since the device is designed with a number of fixing units which typically exhibit only a short local expansion, e.g. 7 cm.

In this case, if the fixing means of a first fixing unit is arranged over a lower end of the fibrous strip of a second fixing unit fixed above the first fixing unit, a continuous measurement by means of the respective fixing unit is guaranteed.

In an optional development each fixing unit comprises a sensitive zone for at least one axis of a three-dimensional space. This enables one direction of movement of the body part to be measured with one sensitive zone. With a number of sensitive zones of different axes a number of different directions of movement of the movement are further able to be measured.

In a preferred development of the invention the fixing loops of the at least one fixing unit are able to be arranged in parallel to a spinal column of the back or on the spinal column of the back. This enables the movements of the spinal column to be well followed by the fiber or fibers, by which a high measurement accuracy can be achieved for a movement of the spinal column.

Preferably the device comprises two fixing units, the two fixing units are arranged in the longitudinal direction in parallel next to each other, in particular the one of the two fixing units is able to be arranged on the left and the other of the two units on the right of a spinal column of a patient. By using two fixing units running in a longitudinal direction in parallel next to each other an increase in the measurement accuracy can be achieved, since as well as a duplication of measurement results in specific directions of movement, torsions can also be detected.

In an optional development for this purpose the respective fixing unit has a single fixing loop, with the fixing loop being formed from a loop-shaped tube elongated in a longitudinal direction of the device made from an expandable material with an adhesive material being provided on the outer side of the tube, the fixing loops formed in this way of the two fixing units being arranged on the expandable fixing support in parallel in a longitudinal direction of the device, a further expandable material expanded in the longitudinal direction of the device being connected by its underside coated with adhesive material to the fixing loops and to the side of the fixing loops to the expandable fixing support. With this optional development the device can be produced in a particularly simple manner which allows industrial production.

In an alternate development the fixing loops of the respective fixing unit are embodied respectively from two expansion strips crossing over each other in the area of the fibrous band. The use of the crossing-over expansion threads for fixing the fibrous strip as fixing loops has the advantage of such a structure being both light since only the expansion threads have a low weight and also of these expansion strips guarantee a secure fixing of the fibrous strips even with greater changes in length of the fixing support. Furthermore when the device is attached to the body part the expansion threads are already expanded, i.e. pretensioned so that for a reduction of the body surface these strips contract and thus make good fixing of the fibrous bands possible. The good fixing allows a good measurement result to be achieved.

If the two fixing units are fixed to an expandable fixing support, the device is well able to follow changes in length of the body part. In addition an increase in the measurement accuracy is evident of the expandable fixing support is only expandable in the longitudinal direction of the device, since this avoids measurement errors from expansions in the transverse direction of the device.

Preferably a further expandable material is fixed with its underside coated with adhesive material to areas of the expandable fixing support running to the left and/or to the right of the respective fibrous strips. This allows both the stability of the device to be increased and the fibrous strip is also able to be better protected against lateral slippage in this way. In particular the expansion threads are connected to the further expandable material.

Preferably a lubrication material to promote the sliding of the fibrous band is arranged in the area of the respective fixing loop below a side pointing towards the body of the respective fibrous strip. This increases the measurement accuracy since measurement errors caused by the fibrous strip tipping when the body part moves are reduced or avoided. A Teflon tape coated on both sides is especially suitable as a lubrication material since its mobility makes it well able to follow the movement of the back and thus not cause any measurement errors.

The invention also relates to a method for production of a device for fixing two fibrous strips in a parallel arrangement by means of an base material comprising a support material and an adhesive material arranged on an underside of the support material on a body part, in which the following steps are executed:
Two loop-shaped tubes are formed from one such that the adhesive material is located on the outer side of the respective tube.
The two loop-shaped tubes are glued in a longitudinal direction in a parallel arrangement to the upper side of the fixing support formed by the base material, with the fixing support being embodied for attaching the device to a body part.
In the longitudinal direction a further expandable material in the form of the base material is attached with the aid of the adhesive material over the fixing support provided with the two loop-shaped tubes.

With this method the device can be produced in a particularly simple manner which allows industrial production.

The invention and its developments will be explained in greater detail with reference to drawings. The individual figures show:

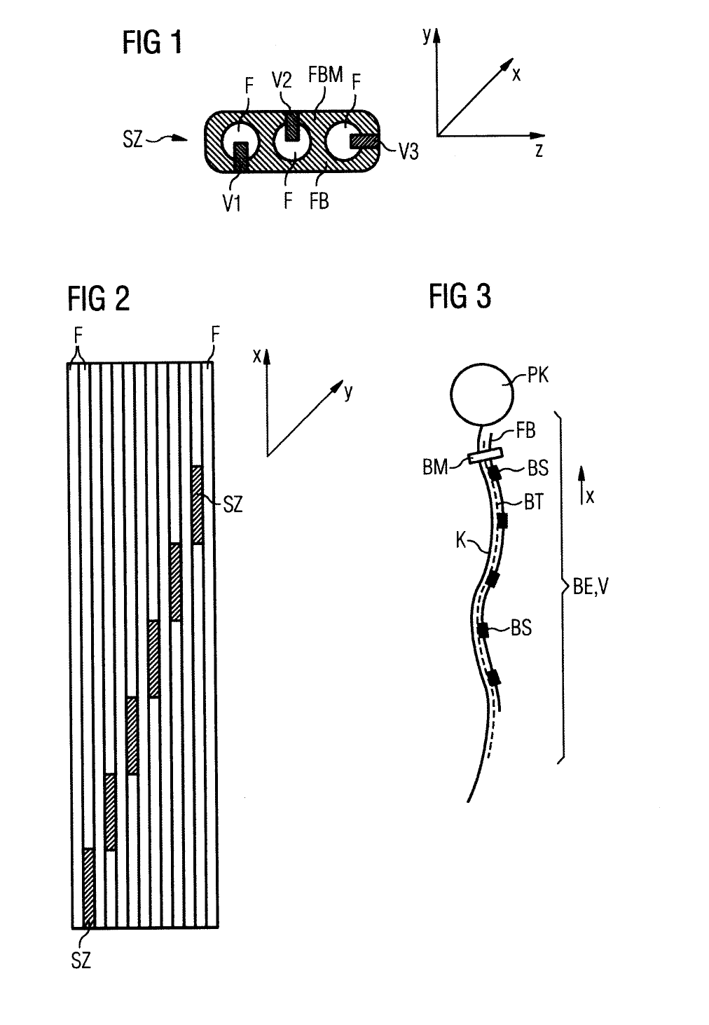

FIG. 1 fibrous strip with three individual fibers for measuring bending in different axes of a three-dimensional space;

FIG. 2 fibrous strip with a number of fibers, with sensitive zones of the fibers being arranged in one axis of the three-dimensional space, but at different positions in the space;

FIG. 3 a first exemplary embodiment with a fixing unit;

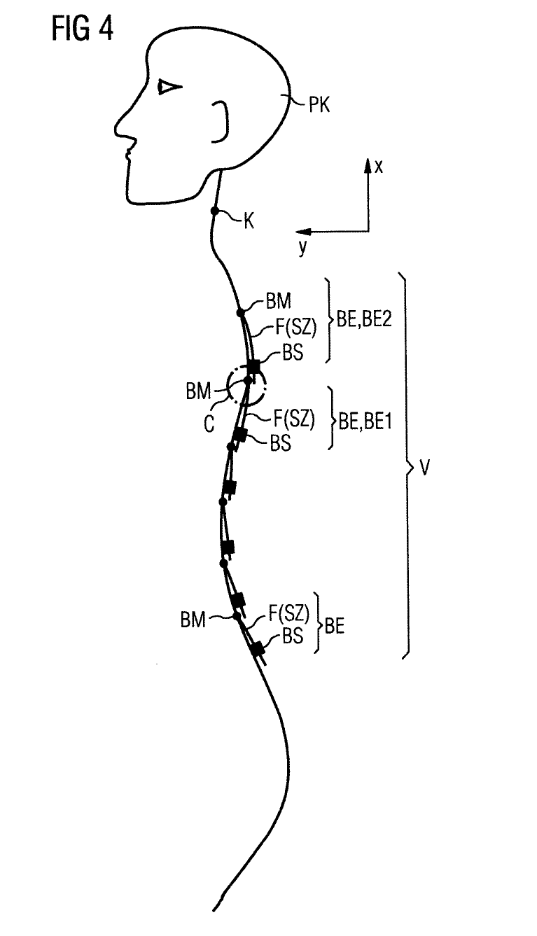

FIG. 4 a second exemplary embodiment with a number of fixing units;

FIG. 5 an expandable support material with an adhesive material on its underside;

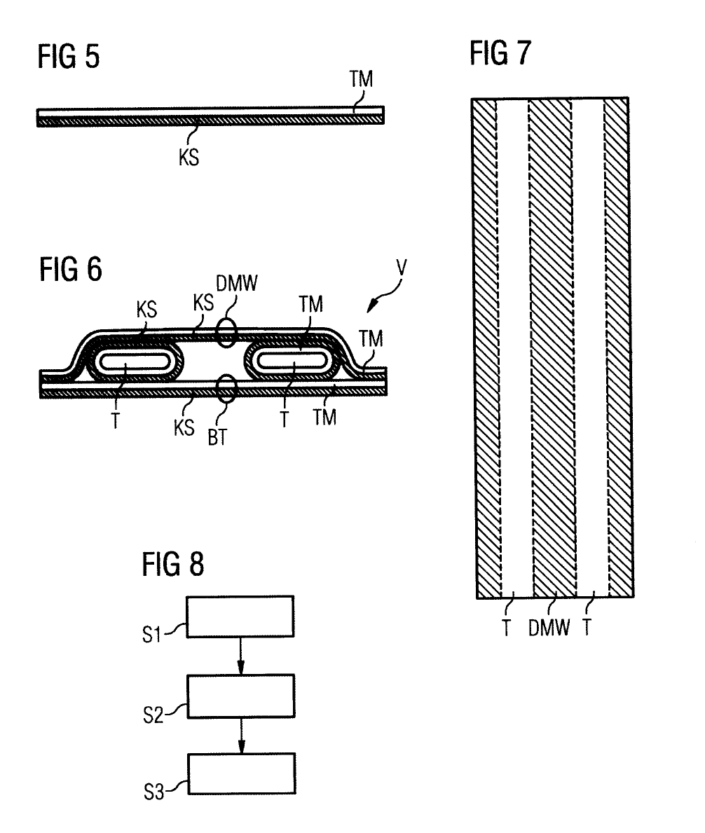

FIG. 6 a further embodiment with two parallel fixing units in cross-section;

FIG. 7 the further embodiment with two parallel fixing units viewed from above;

FIG. 8 a flowchart for production of a device comprising at least two parallel fixing units;

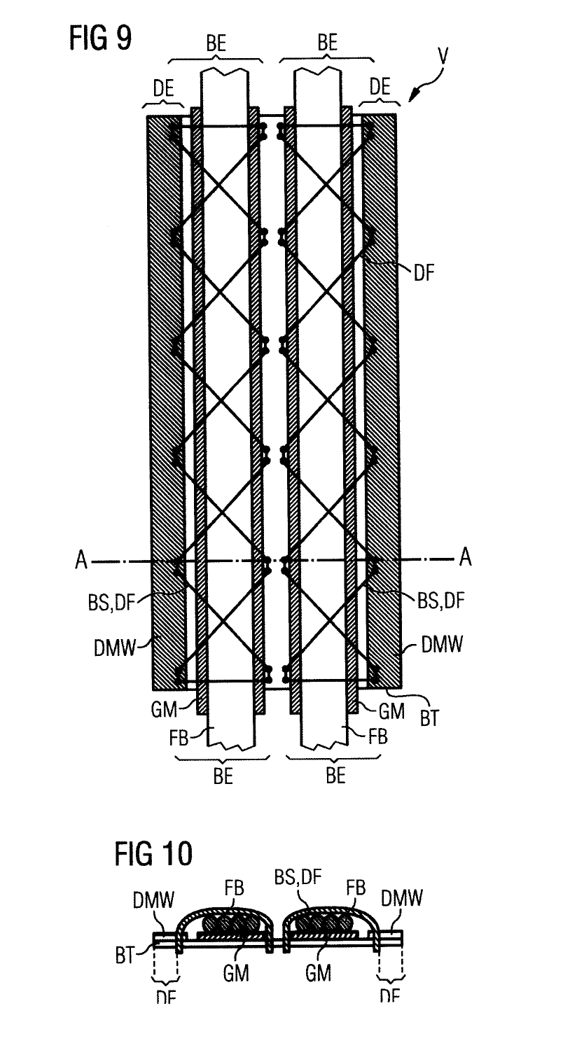

FIG. 9 a last exemplary embodiment of the invention with two parallel fibrous strips which are each fixed with crossing-over expansion threads as fixing loops;

FIG. 10 a cross section of the last exemplary embodiment.

Elements with same function and method of operation are provided in the figures with the same reference symbols.

Fiber optic bending sensors are known from [1] for example. In such sensors a surface of a fiber F is changed at a sensitive zone SZ such that a light beam coupled into the fiber is attenuated by bending the fiber at the sensitive zone. The attenuation changes as a function of the bending of the fiber. FIG. 1 shows a fibrous strip FB comprising three fibers, with the three fibers being mechanically connected to each other by a fibrous strip cover FBM. Each fiber exhibits a mechanical change V1, V2, V3 on a respective fiber surface. Through these mechanical changes, e.g. realized by removal of fiber material and filling with a material other than fiber material, the respective sensitive zone with a bending sensitivity in the axis (x, y, z) of a three-dimensional space is produced.

In FIG. 2 a plurality of fibers F are arranged in a longitudinal direction next to each other as a fibrous strip FB. The fibers each have a mechanical injury, with the respective mechanical injury being arranged at different positions in a axis x of the three-dimensional space, which enables a bending at the different positions of the of the fibrous strip to be detected. For example all mechanical injuries which represent the respective sensitive zone SZ are made on the upper side of the fiber so that a respective bending in a further axis y of the three-dimensional space is able to be determined. The fibrous strip shown here is one possible realization variant, with the present invention being able to be applied to any arrangements of the sensitive zones or injuries for fibers running in parallel in the fibrous strip.

FIG. 3 shows a first exemplary embodiment of the invention. In this figure a person with a head PK and a back can be seen in a schematic representation, with the back representing the body part K to be measured. In this case a form and a deformation of the spinal column over time are to be measured. FIG. 3 further depicts a fixing unit BE comprising a fixing means BM, a number of fixing loops BS and an expandable fixing support BT. The fibrous band FB in accordance with FIG. 2 is typically employed for measurement.

The fixing support BT, shown in FIG. 3 by a dashed line, consists of an expandable material, which on its underside connected to the body part, has adhesive material, i.e. glue. This fixing support is typically a sticking plaster material CureTape® marketed by PhysioTape B. V of The Netherlands, see also http://www.fysiotape.nl/DE/Products/DE 200414892 36 7432938493234.doc/

The fixing support is glued to the back for attaching the device to the latter. The fixing support, as shown by way of example in FIG. 3, can be fully accommodated on the patient's back. Alternatively the fixing support can be accommodated in sections on the back, whereby it connects the fixing loops and the fixing means to the back.

With the fixing unit BE in accordance with the typical embodiment depicted in FIG. 3, the fixing means BM is arranged at the upper end of the spinal column, with the fixing means having the task of fixing the fibrous strip FB to one point of the spinal column to stop it moving. The fixing means is embodied as adhesive tape or as a bracket for example. In the present example the fixing means with the fixing support is attached to the back. In an alternate design the fixing means is fixed directly to the back. The fixing means BM can also fix the fibrous strip at the lower end of the back, so that the end of the fibrous strip pointing upwards on the back can slide in the fixing loops. In general the fixing means can fix the fibrous strip at any given position on the back, e.g. the middle of the fibrous strip approximately in the middle of the back.

Arranged below the fixing means in accordance with FIG. 3 are a number of fixing loops BS which accommodate the fibrous strip FB and make it possible for the fibrous strip to slide in an axis of the three-dimensional space in each case. The surface of the back changes when the back bends and stretches. In such cases the effect of the fixing loops is to make the relatively rigid fibrous strip follow the shape of the spinal column despite bending or stretching of the back. The fixing loops are typically formed from a plastic ring or a rubber ring and are fixed to the spinal column by means of the fixing support BT. In FIG. 3 a device V for fixing at least one fibrous strip comprises precisely one fixing unit BE. In general the fixing means can be attached at any position on the back and to the fibrous strip so that, depending on the arrangement, the fixing loops can be arranged above, below or even above and also below the fixing means. This also applies to the other embodiments.

A further embodiment of the invention is depicted in FIG. 4. In this case the device V comprises a number of fixing units BE for measuring a form of the body part K, i.e. of the back. In such cases each fixing unit comprises a fibrous strip with typically one sensitive zone, which is embodied by means of at least one fiber. Each fixing unit also has a fixing means BM and at least one fixing loop BS. Both the fixing means and also the fixing loop are embodied such that these can be attached directly to the back or the spinal column, e.g. by means of an adhesive material or by means of a suction material. In FIG. 4 the respective fibrous strip is fixed with the fixing means above the fixing loop to the back. For example a fixing unit is attached to every third vertebra. FIG. 4 shows the first and second fixing unit BE1, BE2. To make continuous measurement possible the first and second fixing unit BE1, BE2 of the device V are arranged such that the free end, i.e. the end not fixed by the fixing means, of the fibrous strip of the second fixing unit protrudes beyond the fixing means of a fibrous strip of the adjacent first fixing unit BE1. This can be seen in FIG. 4 in a dotted line circle with a reference symbol C.

The exemplary embodiments below relate to a device V with a number of fixing units BE, with two of the fixing units BE able to be arranged in each case in a longitudinal direction in parallel alongside each other, especially the one of the two fixing units BE to the left and the other of the two fixing unit BE to the right of a spinal column of a patient.

In an exemplary embodiment in accordance with FIGS. 5 to 7 the device V which can be produced in a cost effective manner is shown. An expandable support material TM with an adhesive material KS on its underside serves as the base material, see FIG. 5. For example a commercially-available sticking plaster material, if possible wound onto rolls, can be used for this, such as CureTape® for example. In this case the production process is undertaken in the following steps in accordance with FIG. 8:

Step S1:

From the base material two loop-shaped tubes T, which serve as fixing loops BS, are formed such that the adhesive material KS is located on the outer side of the respective tube. The respective tube typically has a length of half a meter.

Step S2:

The two loop-shaped tubes T are glued in the longitudinal direction in a parallel arrangement to the upper side of the fixing support formed by the base material BT, with there being an adhesive KS on an underside of the fixing support BT. The base material is also the fixing support BT. Since the two tubes each have adhesive material on their outer side, the gluing of the two tubes onto the fixing support can be undertaken by pressing the tubes into the desired orientation.

Step S3:

Attached over the fixing support equipped with the tubes in the longitudinal direction is a further expandable material DMW, which on its underside has an adhesive material K, with its underside on the tubes. The further expandable material can be the base material. In particular the further expandable material is glued to the left and to the right of the tubes with the fixing support.

Steps S1 to S3 produce the device V in accordance with FIG. 6, in which the tube is connected like a sandwich between the fixing support and the further expandable material. FIG. 6 shows a cross-section through the device and FIG. 7 shows a view of the device from above. A length of the device V can be configured depending on the purpose for which it is used. Furthermore the device V can be produced in endless form and cut to the required length by a doctor. FIG. 7 shows an overhead view of the device with the two tubes, which are embodied for guidance of the two fibrous strips.

This exemplary embodiment has not dealt in any greater detail with the manufacturing of the fixing means BM, which is used for fixing the fibrous strips. The fixing means BM of the respective fixing units BE, which are not shown in FIG. 6 or 7, are embodied in the way explained in accordance with FIG. 3.

A last exemplary embodiment is explained in greater detail with reference to FIGS. 9 and 10. FIG. 10 shows a cross-section in the area A-A of the device V from FIG. 9.

The device V in accordance with FIGS. 9 and 10 comprises two fixing units BE, with these being arranged such that the fibrous strips guided in respective fixing units run in a longitudinal direction of the fibrous strips in parallel.

Both fixing units are arranged on a common expandable fixing support BT, which has an adhesive material on its underside with which the device can be glued to the body part. The fixing support is especially a sticking plaster e.g. CureTape®. The fixing loops BS are embodied in this exemplary embodiment from expansion fibers DF, with these expansion fibers being available if stitches are needed as elastic stitch fibers, as typically available from Gold-Zack under the name Strick-Elastic or a rubber band from Rieckmann Kurzwaren GmbH. In this case the fixing loops of the respective fixing units BE are embodied in each case from two crossing-over expansion threads, with these crossing-over expansion threads continuing repeatedly in the longitudinal axis of the device. Between the crossing-over expansion threads and the fixing support is located the respective fibrous strip. In addition a lubrication material GM for promoting the sliding of the fibrous strip, especially a Teflon tape coated on both sides, can be inserted between fibrous strip and fixing support. Furthermore a further expandable material DMW with its underside provided with adhesive material KS can be applied to areas DE of the expandable fixing support BT running to the left and/or right of the respective fibrous strip. In an optional development the expansion threads DF can be connected to the further expandable material DMW. The fixing means BM of the respective fixing unit BE, which are not shown in FIG. 9, can be designed in a similar manner to that explained in accordance with FIG. 3.

It should be noted that in FIGS. 1 to 10 a circuit of the fibers for coupling in a respective light beam and for detecting an attenuated light beam at the end of the respective fiber is not shown.

### LITERATURE REFERENCE

[1] U.S. Pat. No. 5,097,252

1. A device for fixing at least one fibrous strip to a body part, wherein the fibrous strip comprises at least one fiber which is embodied as a fiber optic bending sensor, the device comprising
at least one fixing unit,
wherein the respective fixing unit
comprises a fixing means for attaching a part of the fibrous strip to the body part and
at least one fixing loop for guiding the fibrous strip within an axis of a three-dimensional space, wherein the fixing loop is operable to be fixed to the body part.

2. The device according to claim 1, wherein at least one of
the at least one fixing loop and the fixing means of the at least one fixing unit is attached to an expandable fixing support, with the expandable fixing support operable to be fixed to the body part by an adhesive material.

3. The device according to claim 1, wherein
the device comprises a single fixing unit with a single fixing means and two or more fixing loops,
the body part is a back,
the fixing means is arranged above the fixing loop or in the upper area of the back,
the fixing loops are arranged below one another on the back.

4. The device according to claim 1, wherein
the device comprises two or more fixing units,
each of the fixing units comprises a single fixing means, at least one fixing loop and a fibrous strip with at least one fiber, the fixing units are arranged below one another on the back.

5. The device according to claim 4, wherein
the fixing means of a first fixing unit is arranged above a lower end of the fibrous strip of a second fixing unit attached above the first fixing unit.

6. The device according to claim 4, wherein
each fixing unit comprises a sensitive zone for at least one axis of a three-dimensional space.

7. The device according to claim 1, wherein
the fixing loops of the at least one fixing unit are able to be arranged in parallel to a spinal column of the back or on the spinal column of the back.

8. The device according to claim 1, wherein
the device comprises two fixing units, the two fixing units are arranged in a longitudinal direction in parallel next to one another, especially the one of the two fixing units to the left and the other of the two fixing units to the right of a spinal column of a patient.

9. The device according to claim 8, wherein
the respective fixing unit comprises a single fixing loop, with the fixing loop being embodied from an extended loop-shaped tube in the longitudinal direction made of an expandable material provided with an adhesive material on the outer side of the tube,
the fixing loops of the two fixing units embodied in this way are arranged on an expandable fixing support in parallel in the longitudinal direction of the device,
a further expandable material expanded in the longitudinal direction of the device with its underside coated with adhesive material is connected to the fixing loops and to the side of the fixing loops to the expandable fixing support.

10. The device according to claim 8, wherein
the fixing loops of the respective fixing unit are each embodied from two expansion fibers crossing over each other in the area of the fibrous strip.

11. The device according to claim 10, wherein
the two fixing units are attached to the one expandable fixing support.

12. The device according to claim 11, wherein the expandable fixing support is merely expandable in the longitudinal direction of the device.

13. The device according to claim 10, wherein
attached to areas of the expandable fixing support running on at least one of the left and right of the respective fibrous strips is a further expandable cell material with its underside coated with adhesive material.

14. The device according to claim 13, wherein,
the expansion fibers are connected to the further expandable material.

15. The device according to claim 1, wherein
in the area of the respective fixing loop below a side of the respective fibrous strip pointing towards the body is arranged a lubrication material to promote the sliding of the fibrous strip.

16. The device according to claim 15, wherein,
the lubricant material is a Teflon tape coated on both sides.

17. A method for producing a device for fixing two fibrous strips in a parallel arrangement by means of a base material comprising a support material and an adhesive material arranged on an underside of the support material to a body part, comprising the following steps:
forming two loop-shaped tubes from one loop shape tube such that the adhesive material is located on the outer side of the respective tube,
gluing the two loop-shaped tubes in the longitudinal direction in a parallel arrangement to the upper side of the fixing support formed by the base material, wherein the fixing support is operable to attach the device to a body part,
attaching in the longitudinal direction a further expandable material in the form of the base material with the aid of the adhesive material, above the fixing support provided with the two loop-shaped tubes.

18. A method for fixing at least one fibrous strip to a body part, wherein the fibrous strip comprises at least one fiber which is embodied as a fiber optic bending sensor, the method comprising the steps of
attaching a part of the fibrous strip to the body part by fixing means, and
guiding the fibrous strip within an axis of a three-dimensional space by at least one fixing loop, wherein the fixing loop is operable to be fixed to the body part.

19. The method according to claim 18,
Comprising the step of attaching at least one of
the at least one fixing loop and the fixing means of the at least one fixing unit to an expandable fixing support with the expandable fixing support operable to be fixed to the body part by an adhesive material.

20. The device according to claim 1,
wherein at least one of
the at least one fixing loop and the fixing means of the at least one fixing unit is attached to a sticking plaster material operable to be fixed to the body part by an adhesive material.
